(19) United States
(12) Patent Application Publication
(10) Pub. No.: US 2025/0032458 A1
(43) Pub. Date: Jan. 30, 2025

(54) TREATMENT OF CANCER WITH AN FGFR KINASE INHIBITOR

(71) Applicant: Kinnate Biopharma Inc., San Diego, CA (US)

(72) Inventors: Aleksandra FRANOVIC, Del Mar, CA (US); Angie VASSAR, San Diego, CA (US); Nichol MILLER, Poway, CA (US); Ken KOBAYASHI, Rancho Santa Fe, CA (US); Richard Thomas WILLIAMS, Newbury Park, CA (US); John TYHONAS, San Diego, CA (US); Robert KANIA, Del Mar, CA (US); Jason M. COX, Rancho Santa Fe, CA (US); Neolito TIMPLE, San Diego, CA (US); Eric S. MARTIN, Del Mar, CA (US); Eric A. MURPHY, San Marcos, CA (US)

(21) Appl. No.: 18/717,345

(22) PCT Filed: Dec. 7, 2022
(86) PCT No.: PCT/US2022/081059

§ 371 Date: Jun. 6, 2024

Related U.S. Application Data
(60) Provisional application No. 63/287,456, filed on Dec. 8, 2021.

(51) Int. Cl. A61K 31/4184; A61K 45/06; A61P 35/00

(52) U.S. Cl. CPC A61K 31/4184; A61K 45/06; A61P 35/00

(57) ABSTRACT

Provided herein are compositions and methods for the treatment of a cancer. Said compositions comprise an FGFR kinase inhibitor. Some embodiments comprise combination therapy featuring the FGFR kinase inhibitor with at least one oncology therapeutic agent.

### CROSS-REFERENCE TO RELATED APPLICATION

This application claims the benefit of U.S. Patent Application No. 63/287,456, filed on Dec. 8, 2021, which is hereby incorporated by reference in its entirety.

### BACKGROUND OF THE INVENTION

Fibroblast growth factor receptors (FGFRs) are a subfamily of receptor tyrosine kinases (RTKs) that bind to members of the fibroblast growth factor family of proteins. Deregulation of the fibroblast growth factor/FGF receptor network occurs frequently in tumors. Accordingly, therapies that target aberrant FGFR kinase activity are desired for use in the treatment of cancer and other disorders. One such modulator of FGFR kinase is 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

### BRIEF SUMMARY OF THE INVENTION

One embodiment provides a method of treating a cancer in a patient in need thereof, comprising administering to the patient 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, wherein the cancer is selected from bladder cancer, urinary bladder carcinoma, urothelial carcinoma, urothelial cancer, renal cell carcinoma, prostate cancer, double negative prostate, castration-resistant prostate cancer, gastric carcinoma, gastric cancer, gastroesophageal junction adenocarcinoma, hepatocellular carcinoma, cholangiocarcinoma, intrahepatic cholangiocarcinoma, pancreatic adenocarcinoma, pancreatic cancer, breast cancer, HER2(−)/ER(+) breast cancer, HER2(−)/ER(+)/PR(+) breast cancer, non-Hodgkin lymphoma, acute myeloid leukemia, myeloproliferative neoplasm, polycythemia vera, essential thrombocythemia, primary myelofibrosis, multiple myeloma, glioblastoma, glioma, astrocytoma, anaplastic astrocytoma, medulloblastoma, oligodendroglioma, anaplastic oligodendroglioma, meningioma, lung cancer, or non-small cell lung cancer. Another embodiment provides the method, wherein the cancer is characterized as having an oncogenic FGFR alteration. Another embodiment provides the method, wherein the cancer is characterized as having an oncogenic FGFR2 alteration. Another embodiment provides the method, wherein the cancer is characterized as having an oncogenic FGFR3 alteration.

### BRIEF DESCRIPTION OF THE DRAWINGS

FIGS. 1A-1C illustrate antitumor activity of Compound 1 in a RT-112 FGFR3-driven human cancer cell line-derived xenograft model.

FIGS. 2A-2C illustrate antitumor activity of Compound 1 in a SNU-16 FGFR2-driven human cancer cell line-derived xenograft model.

FIG. 3 provides a flowchart of the BOIN Study Design.

### INCORPORATION BY REFERENCE

All publications, patents, and patent applications mentioned in this specification are hereby incorporated by reference for the specific purposes identified herein.

### DETAILED DESCRIPTION

### Certain Terminology

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Pharmaceutically acceptable salt” includes both acid and base addition salts. A pharmaceutically acceptable salt of the heterocyclic FGFR kinase inhibitor described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made. The term “treating”, as used herein, unless otherwise indicated, means reversing, alleviating, inhibiting the progress of, or preventing the disorder or condition to which such term applies, or one or more symptoms of such disorder or condition. In some embodiments, the term “treating” includes slowing or delaying the progression of the disease or disorder to which the term is applied. Additionally, in some embodiments, the term “treating” is applied to one or more of the complications resulting from the disease or disorder to which the term is applied. The term “treatment”, as used herein, unless otherwise indicated, refers to the act of treating as “treating” is defined immediately above.

The term “tumor,” or “cancer” as used herein, and unless otherwise specified, refers to a neoplastic cell growth, and includes pre-cancerous and cancerous cells and tissues. Tumors usually present as a lesion or lump. As used herein, “treating” a tumor means that one or more symptoms of the disease, such as the tumor itself, vascularization of the tumor, or other parameters by which the disease is characterized, are reduced, ameliorated, inhibited, placed in a state of remission, or maintained in a state of remission. “Treating” a tumor also means that one or more hallmarks of the tumor may be eliminated, reduced or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries.

The term “refractory” or “refractory to therapy” indicates that the patients have never responded to therapy.

The term “relapsed” or “relapsed after therapy” indicates that patients, after initially responding to prior therapy, have progressive disease due to acquired resistance and/or intolerance.

The term “resistance to therapy” or “acquired resistance to therapy” indicates the patients, after initially responding to prior therapy, have progressive disease due to clinical or molecular resistance to the therapy. The acquired resistance can result from emergence of resistant mutations in the molecular target of the therapy, or in the development of physiological functions such as efflux pumps.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

Other aspects, advantages, and features of the invention will become apparent from the detailed description below.

### Fibroblast Growth Factor Receptor

Fibroblast growth factor receptor (FGFR) are receptor tyrosine kinases that regulate diverse physiological and pathological processes ranging from embryonic development to tumorigenesis. FGFR family members (FGFR1, FGFR2, FGFR3, FGFR4) are transmembrane proteins that contain extracellular ligand-binding domain and an intracellular tyrosine-kinase domain. In the absence of the fibroblast growth factor (FGF) ligand, non-phosphorylated FGFR kinases remain in an inactive conformation. Binding of FGF ligands leads to receptor dimerization and auto-phosphorylation, followed by subsequent activation of downstream signaling pathways, such as the rat sarcoma viral oncogene homolog/mitogen-activated protein kinase (RAS-MAPK), phosphatidylinositol 3-kinase/protein kinase B (PI3K-AKT), and phospholipase C γ/protein kinase C (PLCγ-PKC) axes, which regulate cell proliferation, survival, and migration (Babina & Turner 2017, Katoh 2019).

Oncogenic FGFR gene alterations, which are observed in approximately 7% of all human cancers, typically present as activating point mutations, small intragenic deletions, genomic amplifications, or chromosomal rearrangements/fusions (Cerami et al. 2012, Gao et al., 2013, Helsten et al., 2016), resulting in aberrant signaling and driving tumorigenesis. Consequently, dysregulated FGFR signaling promotes the proliferation, survival, and development of drug resistance in tumor cells (Babina & Turner 2017, Katoh 2019). FGFR2 gene fusions and FGFR3 activating alterations, in particular, are predicted drivers in 10-20% of cholangiocarcinoma and 20-35% of urothelial cancers, respectively (Katoh 2019, Krook et al. 2020). Consistently, pharmacological inhibition of FGFR has pronounced anti-proliferative and anti-tumor effects in preclinical models of FGFR-dependent human cancers, supporting the clinical development of FGFR inhibitors (Hall et al. 2016, Perera et al. 2017, Goyal et al. 2019, Liu et al. 2020, Sootome et al. 2020).

Three FGFR inhibitors, erdafitinib, pemigatinib, and infigratinib, have recently been approved by the United States Food and Drug Administration for the treatment of patients with advanced or metastatic FGFR2- and FGFR3-driven cancers (Loriot et al., 2019, Abou-Alfa et al. 2020, Jayle et al. 2021, BALVERSA® Package Insert [PI], PEMAZYRE™ PI, TRUSELTIQ™ PI). A major limitation of the currently approved and clinical-stage FGFR inhibitors is the emergence of secondary, on-target gene alterations that limit duration of response (Goyal et al. 2017, Goyal et al. 2019, Silverman et al. 2021, Varghese et al. 2021). Mutations in the FGFR kinase domain at key gatekeeper residues lead to steric hindrance within the adenosine triphosphate (ATP) binding pocket and block access of ATP-competitive FGFR inhibitors. Mutations in gatekeeper residues in respective FGFRs (FGFR1-V561, FGFR2-V565F (also known as FGFR2-V564F), FGFR3-V555M, FGFR4-V550L) have been demonstrated to confer resistance to reversible, type I pan-FGFR inhibition (Dai et al. 2019). FGFR2 kinase domain single-nucleotide variants corresponding to known gatekeeper and activating mutations have been identified in patients that progressed on FGFR inhibitor treatment and have been demonstrated to constitute an acquired resistance mechanism in intrahepatic cholangiocarcinoma (Goyal et al. 2017, Goyal et al. 2019). Analogous activating mutations in FGFR3 have been detected in patient samples and exhibit resistance in preclinical models (Patani et al. 2016).

### Heterocyclic FGFR Kinase Inhibitor

The heterocyclic FGFR kinase inhibitor described herein refers to Compound 1 having the structure below, and the chemical name 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

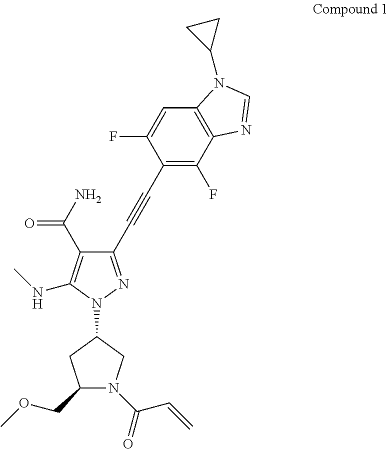 [SMILES: C=CC(=O)N1C[C@@H](n2nc(C#Cc3c(F)cc4c(ncn4C4CC4)c3F)c(C(N)=O)c2NC)C[C@@H]1COC]

Compound 1 is an irreversible small molecule FGFR kinase inhibitor. Throughout this disclosure when reference is made to a heterocyclic FGFR kinase inhibitor, or pharmaceutically acceptable salts or solvates thereof, the reference is to Compound 1, or pharmaceutically acceptable salts or solvates thereof.

### Cancer and Methods of Treatment

In one embodiment is a method for inhibiting FGFR kinase enzyme comprising contacting the enzyme with Compound 1, or pharmaceutically acceptable salts or solvates thereof, as disclosed herein. In certain aspects, disclosed herein is a method of treating a cancer in an individual in need thereof, comprising administering an effective amount of a heterocyclic FGFR kinase inhibitor described herein to the individual. In certain aspects, disclosed herein is a heterocyclic FGFR kinase inhibitor for use in treating a cancer. In certain aspects, disclosed herein is a heterocyclic FGFR kinase inhibitor described herein for use in preparation of a medicament for treating a cancer.

One embodiment provides a method of treating a cancer in a patient in need thereof, comprising administering to the patient 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof. One embodiment provides a method of treating a cancer in a patient in need thereof, comprising administering to the patient a pharmaceutical composition comprising 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, and at least one pharmaceutically acceptable excipient. Another embodiment provides the method, wherein the cancer is characterized as having an oncogenic FGFR alteration. Another embodiment provides the method, wherein the cancer is characterized as having an oncogenic FGFR2 alteration. Another embodiment provides the method, wherein the cancer is characterized as having an oncogenic FGFR3 alteration. Another embodiment provides the method, wherein the FGFR alteration is selected from the group consisting of:
FGFR2 [K660M];
FGFR2 [K659M];
FGFR2 [L618V];
FGFR2 [L617V];
FGFR2 [N550H];
FGFR2 [N549H];
FGFR2 [N550K];
FGFR2 [N549K];
FGFR2 [V565F];
FGFR2 [V564F];
FGFR2 [N550S/T];
FGFR2 [N549S/T];
FGFR3 [G697C];
FGFR3 [V555M];
FGFR3 [K650E];
FGFR3 [N540K/S];
FGFR3 [K650M]; and
FGFR1 [V561M]; or a combination thereof.

Another embodiment provides the method, wherein the FGFR alteration is selected from at least one alteration disclosed in Table 1.

TABLE 1

| Alteration | Funtional | | | | |
|---|---|---|---|---|---|
| Type | Description | FGFR2 Alteration | | | |
| Fusion | Activating via | FGFR2- | FGFR2- | FGFR2- | FGFR2- |
| | upregulation and/ | ACLY | DNAJC12 | NOL4 | SORBS1 |
| | or ligand- | FGFR2- | FGFR2- | FGFR2- | FGFR2- |
| | independent | AFF3 | DSP | NRAP | SPERT |
| | dimerization | FGFR2- | FGFR2- | FGFR2- | FGFR2- |
| | | AFF4 | EEA1 | NRBF2 | SPICE1 |
| | | FGFR2- | FGFR2- | FGFR2- | FGFR2- |
| | | AHCYL1 | EIF4ENIF1 | OFD1 | STRN4 |
| | | FGFR2- | FGFR2- | FGFR2- | FGFR2- |
| | | ARHGAP22 | ERC1 | OPTN | TACC1 |
| | | FGFR2- | FGFR2- | FGFR2- | FGFR2- |
| | | ARHGAP24 | FAM160B1 | PAH | TACC2 |
| | | FGFR2- | FGFR2- | FGFR2- | FGFR2- |
| | | ATAD2 | FAM67A | PAWR | TACC3 |
| | | FGFR2- | FGFR2- | FGFR2- | FGFR2- |
| | | ATF2 | FILIP1 | PIBF1 | TBC1D1 |
| | | FGFR2- | FGFR2- | FGFR2- | FGFR2- |
| | | BICC1 | GAB2 | POC1B | TCTN3 |
| | | FGFR2- | FGFR2- | FGFR2- | FGFR2- |
| | | BICD1 | GOPC | POF1B | TFEC |
| | | FGFR2- | FGFR2- | FGFR2- | FGFR2- |
| | | CASP7 | INSC | PPHLN1 | TRIM8 |
| | | FGFR2- | FGFR2- | FGFR2- | FGFR2- |
| | | CCDC158 | KCTD1 | PXN | TTC28 |
| | | FGFR2- | FGFR2- | FGFR2- | FGFR2- |
| | | CCDC170 | KIAA1217 | RABGAP1L | TXLNB |
| | | FGFR2- | FGFR2- | FGFR2- | FGFR2- |
| | | CCDC6 | KIAA1598 | RASSF4 | USH2A |
| | | FGFR2- | FGFR2- | FGFR2- | FGFR2- |
| | | CDC42BPB | KIAA1967 | ROBO2 | USP10 |
| | | FGFR2- | FGFR2- | FGFR2- | FGFR2- |
| | | CEP128 | MACF1 | RPAP3 | VCL |
| | | FGFR2- | FGFR2- | FGFR2- | FGFR2- |
| | | CIT | MATR3 | SFI1 | WAC |
| | | FGFR2- | FGFR2- | FGFR2- | FGFR2- |
| | | COL16A1 | MCU | SHROOM3 | WDHD1 |
| | | FGFR2- | FGFR2- | FGFR2- | FGFR2- |
| | | CTNNA3 | MGEA5 | SLMAP | ZMYM4 |
| | | FGFR2- | FGFR2- | FGFR2- | FGFR2- |
| | | DBP | NEDD4L | SOGA1 | ZNF521 |

| | | |
|---|---|---|
| Insertion- | Extracellular | H167_N173del |
| Deletion | domain | P170_K176del |
| | activating | P256_G261delinsR |
| | mutation | P263_A266del |
| | | T268_D273delinsS |
| | | V280_K292del |
| | | P286_K292del |
| | | I288_E295delinsT |
| | | W290_I291delinsC |
| Missense | Activating Loop | K659M |
| | | K659N |
| | Extracellular | D101Y |
| | domain | R203C |
| | activating | R251Q |
| | mutation | S252W |
| | | P253R |
| | | F276C |
| | | W290R |
| | | T341P |
| | | S372C |
| | | Y375C |
| | Gatekeeper | V564F |
| | | V564I |
| | | V564L |
| | Molecular | N549H |
| | Brake/Regulatory | N549K |
| | Triad | N549S |
| | | N549T |
| | | E565G |
| | | K641N |
| | | K641R |
| | Other kinase | K526E |
| | domain | M535I |
| | activating | M537I |
| | mutations | M538I |
| | | I547V |
| | | V562L |
| | | L617F |
| | | L617M |
| | | L617V |
| | | R678G |
| | | H682L |
| | | K714R |
| | | E731K |
| | Transmembrane | Y381D |
| | domain | C382R |
| | activating | M391R |
| | mutation | |
| Duplication | Extracellular | S267_D273dup |
| | domain | |
| | activating | |
| | mutation | |
| Amplifi- | Activating via | Amplification |
| cation | upregulation | |

Note:
Nomenclature based on FGFR2 IIIc splice variant; add 1 to amino acid position for corresponding FGFR2 IIIb splice variant (e.g., N549K → N550K).

Another embodiment provides the method, wherein the oncogenic FGFR alteration is FGFR2 amplification, FGFR2 fusion or rearrangement, FGFR2 insertion-deletion mutation, FGFR3 fusion or rearrangement, FGFR3-TACC3, or FGFR3-BAIAP2L1.

One embodiment provides the method of treating cancer wherein the cancer is selected from bladder cancer, urinary bladder carcinoma, urothelial carcinoma, urothelial cancer, renal cell carcinoma, prostate cancer, double negative prostate, castration-resistant prostate cancer, gastric carcinoma, gastric cancer, gastroesophageal junction adenocarcinoma, hepatocellular carcinoma, cholangiocarcinoma, intrahepatic cholangiocarcinoma, pancreatic adenocarcinoma, pancreatic cancer, breast cancer, HER2(−)/ER(+) breast cancer, HER2(−)/ER(+)/PR(+) breast cancer, non-Hodgkin lymphoma, acute myeloid leukemia, myeloproliferative neoplasm, polycythemia vera, essential thrombocythemia, primary myelofibrosis, multiple myeloma, glioblastoma, glioma, astrocytoma, anaplastic astrocytoma, medulloblastoma, oligodendroglioma, anaplastic oligodendroglioma, meningioma, lung cancer, or non-small cell lung cancer.

Another embodiment provides the method of treating cancer wherein the tumor is characterized by the presence of at least one FGFR2 or FGFR3 gene alteration. Another embodiment provides the method of treating cancer wherein the tumor is characterized by the presence of FGFR2 and FGFR3 gene alterations. Another embodiment provides the method of treating cancer wherein the tumor is characterized by the presence of at least one FGFR1, FGFR2 or FGFR3 gene alteration. Another embodiment provides the method of treating cancer wherein the tumor is characterized by the presence of any FGFR gene alteration. Another embodiment provides the method of treating cancer wherein the patient is selected due to a FGFR1, FGFR2 or FGFR3 gene alteration as detected by an FDA-approved test. Another embodiment provides the method of treating cancer wherein the patient is selected due to a FGFR2 or FGFR3 fusion or rearrangement as detected by an FDA-approved test. Another embodiment provides the method of treating cancer wherein the patient is selected due to a FGFR2 fusion or rearrangement as detected by an FDA-approved test. Another embodiment provides the method of treating cancer wherein the patient is selected due to a FGFR3 fusion or rearrangement as detected by an FDA-approved test.

One embodiment provides the method of treating cancer wherein the cancer is selected from gastric carcinoma, gastric cancer, or gastroesophageal junction adenocarcinoma.

One embodiment provides the method of treating cancer wherein the cancer is selected from bladder cancer, urinary bladder carcinoma, urothelial carcinoma, or urothelial cancer. One embodiment provides the method of treating cancer wherein the cancer is selected from cholangiocarcinoma, intrahepatic cholangiocarcinoma, pancreatic adenocarcinoma, pancreatic cancer. One embodiment provides the method of treating cancer wherein the cancer is selected from gastric carcinoma, gastric cancer, or gastroesophageal junction adenocarcinoma and the patient is selected due to a FGFR2 or FGFR3 fusion or rearrangement as detected by an FDA-approved test.

One embodiment provides the method of treating cancer wherein the cancer is selected from bladder cancer, urinary bladder carcinoma, urothelial carcinoma, or urothelial cancer and the patient is selected due to a FGFR2 or FGFR3 fusion or rearrangement as detected by an FDA-approved test. One embodiment provides the method of treating cancer wherein the cancer is selected from cholangiocarcinoma, intrahepatic cholangiocarcinoma, pancreatic adenocarcinoma, pancreatic cancer and the patient is selected due to a FGFR2 or FGFR3 fusion or rearrangement as detected by an FDA-approved test. One embodiment provides the method of treating cancer wherein the cancer is selected from prostate cancer, double negative prostate, or castration-resistant prostate cancer. One embodiment provides the method of treating cancer wherein the cancer is selected from breast cancer, HER2(−)/ER(+) breast cancer, or HER2(−)/ER(+)/PR(+) breast cancer. One embodiment provides the method of treating cancer, wherein the cancer is selected from non-Hodgkin lymphoma. One embodiment provides the method of treating cancer wherein the cancer is selected from acute myeloid leukemia. One embodiment provides the method of treating cancer wherein the cancer is selected from multiple myeloma. One embodiment provides the method of treating cancer wherein the cancer is selected from myeloproliferative neoplasms including polycythemia vera, essential thrombocythemia, and primary myelofibrosis. One embodiment provides the method of treating cancer wherein the cancer is selected from glioblastoma, glioma, astrocytoma, anaplastic astrocytoma, medulloblastoma, oligodendroglioma, anaplastic oligodendroglioma, or meningioma. One embodiment provides the method of treating cancer wherein the cancer is selected from lung cancer, or non-small cell lung cancer. One embodiment provides the method of treating cancer wherein the cancer is renal cell carcinoma. One embodiment provides the method of treating cancer wherein the cancer is hepatocellular carcinoma.

One embodiment provides the method of treating cancer wherein the cancer is bladder cancer. One embodiment provides the method of treating cancer wherein the cancer is urinary bladder carcinoma. One embodiment provides the method of treating cancer wherein the cancer is urothelial carcinoma. One embodiment provides the method of treating cancer wherein the cancer is urothelial cancer. One embodiment provides the method of treating cancer wherein the cancer is cholangiocarcinoma. One embodiment provides the method of treating cancer wherein the cancer is intrahepatic cholangiocarcinoma.

One embodiment provides the method of treating cancer wherein the cancer is bladder cancer and the patient is selected due to a FGFR2 or FGFR3 fusion or rearrangement as detected by an FDA-approved test. One embodiment provides the method of treating cancer wherein the cancer is urinary bladder carcinoma and the patient is selected due to a FGFR2 or FGFR3 fusion or rearrangement as detected by an FDA-approved test. One embodiment provides the method of treating cancer wherein the cancer is urothelial carcinoma and the patient is selected due to a FGFR2 or FGFR3 fusion or rearrangement as detected by an FDA-approved test. One embodiment provides the method of treating cancer wherein the cancer is urothelial cancer and the patient is selected due to a FGFR2 or FGFR3 fusion or rearrangement as detected by an FDA-approved test. One embodiment provides the method of treating cancer wherein the cancer is cholangiocarcinoma and the patient is selected due to a FGFR2 or FGFR3 fusion or rearrangement as detected by an FDA-approved test. One embodiment provides the method of treating cancer wherein the cancer is intrahepatic cholangiocarcinoma and the patient is selected due to a FGFR2 or FGFR3 fusion or rearrangement as detected by an FDA-approved test.

Another embodiment provides the method, wherein the cancer is metastatic. Another embodiment provides the method, wherein the method is adjuvant therapy following surgical resection. Another embodiment provides the method, wherein the method is neo-adjuvant therapy before surgical resection. Another embodiment provides the method, wherein the patient has relapsed after prior therapy. Another embodiment provides the method, wherein the patient has acquired resistance to prior therapy. Another embodiment provides the method, wherein the patient is refractory to therapy. Another embodiment provides the method, wherein the 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, is administered orally. Another embodiment provides the method, wherein the composition comprising 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, or pharmaceutically acceptable salt or solvate thereof, and at least one pharmaceutically acceptable excipient, is administered orally. Another embodiment provides the method, wherein the oral administration occurs every other day, once per day, twice per day, or three times per day.

One embodiment provides a method of treating a cancer in a patient in need thereof, comprising administering to the patient:
(a) a composition comprising 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, and
(b) at least one oncology therapeutic selected from an mTOR inhibitor, a MAPK/PI3K inhibitor, an immune checkpoint inhibitor, an EGFR kinase inhibitor or antibody, a HER2 kinase inhibitor, an estrogen receptor antagonist, an androgen receptor antagonist, a CDK kinase inhibitor, an ALK receptor tyrosine kinase inhibitor, a ROS receptor tyrosine kinase inhibitor, a NTRK receptor tyrosine kinase inhibitor, or a chemotherapy regimen.

Another embodiment provides the method, wherein the at least one oncology therapeutic is an mTOR inhibitor. Another embodiment provides the method, wherein the mTOR inhibitor is rapamycin. Another embodiment provides the method, wherein the at least one oncology therapeutic is a MAPK/PI3K inhibitor. Another embodiment provides the method, wherein the MAPK/PI3K inhibitor is binimetinib or copanlisib. Another embodiment provides the method, wherein the at least one oncology therapeutic is a HER2 kinase inhibitors. Another embodiment provides the method, wherein the HER2 inhibitor is lapatinib. Another embodiment provides the method, wherein the at least one oncology therapeutic is an immune checkpoint inhibitor. Another embodiment provides the method, wherein the immune checkpoint inhibitor is a CTLA-4 inhibitor, a PD-1 inhibitor, or a PD-L1 inhibitor. Another embodiment provides the method, wherein the CTLA-4 inhibitor is ipilimumab. Another embodiment provides the method, wherein the PD-1 inhibitor is spartalizumab, nivolumab, atezolizumab, pembrolizumab, or cemiplimab. Another embodiment provides the method, wherein the PD-L1 inhibitor is atezolizumab, avelumab, or durvalumab. Another embodiment provides the method, wherein the at least one oncology therapeutic is a CDK inhibitor. Another embodiment provides the method, wherein the CDK inhibitor is a CDK4/6 inhibitor. Another embodiment provides the method, wherein the CDK4/6 inhibitor is palbociclib, abemaciclib, or ribociclib. Another embodiment provides the method, wherein the at least one oncology therapeutic is an EGFR kinase inhibitor or antibody. Another embodiment provides the method, wherein the EGFR kinase inhibitor is nazartinib, gefitinib, erlotinib, afatinib, brigatinib, icotinib, neratinib, osimertinib, dacomitinib, or lapatinib. Another embodiment provides the method, wherein the EGFR antibody is cetuximab, panitumumab, zalutumumab, nimotuzumab, or matuzumab. Another embodiment provides the method, wherein the at least one oncology therapeutic is an estrogen receptor antagonist. Another embodiment provides the method, wherein the estrogen receptor antagonist is fulvestrant. Another embodiment provides the method, wherein the at least one oncology therapeutic is an androgen receptor antagonist. Another embodiment provides the method, wherein the androgen receptor antagonist is enzalutamide. Another embodiment provides the method, wherein the at least one oncology therapeutic is selected from an ALK receptor tyrosine kinase inhibitor, a ROS receptor tyrosine kinase inhibitor, or a NTRK receptor tyrosine kinase inhibitor. Another embodiment provides the method, wherein the at least one oncology therapeutic is a chemotherapy regimen. Another embodiment provides the method, wherein the chemotherapy regimen is a cisplatin regimen, a gemcitabine regimen, or a FOLFOX regimen.

### Pharmaceutical Compositions

In certain embodiments, the heterocyclic FGFR kinase inhibitor described herein is administered as a pure chemical. In other embodiments, the heterocyclic FGFR kinase inhibitor described herein is combined with a pharmaceutically suitable or acceptable carrier (also referred to herein as a pharmaceutically suitable or acceptable excipient, a physiologically suitable or acceptable excipient, or a physiologically suitable or acceptable carrier) selected on the basis of a chosen route of administration and standard pharmaceutical practice.

Provided herein is a pharmaceutical composition comprising the heterocyclic FGFR kinase inhibitor as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a method of preparing a pharmaceutical composition comprising mixing the heterocyclic FGFR kinase inhibitor as described herein, or a stereoisomer, pharmaceutically acceptable salt, hydrate, or solvate thereof, and a pharmaceutically acceptable carrier.

Provided herein is the method wherein the pharmaceutical composition is administered orally. Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

Provided herein is the method wherein the pharmaceutical composition is administered by injection. In some embodiments, the heterocyclic FGFR kinase inhibitor as described herein, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising the heterocyclic FGFR kinase inhibitor as described herein, or a stereoisomer, pharmaceutically acceptable salt, hydrate, or solvate thereof, differs depending upon the subject or patient's (e.g., human) condition. In some embodiments, such factors include general health status, age, and other factors. Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

### EXAMPLES

These examples are provided for illustrative purposes only and not to limit the scope of the claims provided herein.

Compound 1, a novel, next-generation, irreversible, small molecule pan-FGFR inhibitor, was structurally designed to inhibit clinically observed secondary mutations known to drive resistance to the approved FGFR inhibitors. This study evaluated the biochemical inhibitory activity of Compound 1 against a panel of wild type and mutant FGFR kinases, as well as selectivity against the rest of the kinome.

### Example 1: FGFR Kinase Inhibitory Activity of Compound 1

Compound 1 was tested in biochemical assays to assess activity against both wild type and mutant FGFR kinase family members in multiple independent experiments. Mobility shift assays were performed across a panel of purified FGFR kinase enzymes. Table 2 lists the mean potency of Compound 1 inhibition against the panel of enzymes. Wild type FGFR1, FGFR2, FGFR3 and FGFR4 demonstrated IC₅₀ values of 3.90 nM, 5.25 nM, 9.70 nM, and 4.91 nM, respectively. Kinase domain mutations in the respective gatekeeper residues FGFR1-V561M, FGFR2-V565F (also known as FGFR2-V5654F), and FGFR3-V555M demonstrated IC₅₀ values of 62.98 nM, 20.81 nM, and 24.27 nM, respectively. FGFR2-N550H (also known as FGFR2-N549H) molecular brake and FGFR3-K650M activating mutations demonstrated IC₅₀ values of 22.80 nM and 4.63 nM, respectively.

TABLE 2

| Enzyme | IC₅₀ (nM) | SEM | n |
|---|---|---|---|
| FGFR1 | 3.90 | 0.46 | 15 |
| FGFR1 [V561M] | 62.98 | 23.31 | 11 |
| FGFR2 | 5.25 | 1.51 | 7 |
| FGFR2 [N549H] AKA | 22.80 | 2.10 | 12 |
| FGFR2 [N550H] | | | |
| FGFR2 [V564F] AKA | 20.81 | 1.64 | 5 |
| FGFR2 [V565F] | | | |
| FGFR3 | 9.70 | 2.61 | 6 |
| FGFR3 [V555M] | 24.27 | 2.61 | 15 |
| FGFR3 [K650M] | 4.63 | 0.37 | 11 |
| FGFR4 | 4.91 | 0.73 | 5 |

AKA = also known as;
FGFR = fibroblast growth factor receptor;
FGFR = fibroblast growth factor receptor;
IC₅₀ = half-maximal inhibitory concentration;
n = number;
SEM = standard error of the mean

Broad kinome selectivity of Compound 1 was tested by mobility shift assay (MSA). Compound 1 at a concentration of 1 μM was screened against a panel of 321 kinases. Table 3 lists any kinases that exhibited >40% inhibition. Only 2 non-FGFR kinases in this panel exhibited >40% inhibition, including TNK1 and LOK (AKA serine threonine kinase 10 [STK10]) at 89.5% and 64.6%, respectively.

TABLE 3

| | % Inhibition |
|---|---|
| Kinase | 1 μM |
| FGFR3 [K650E] | 99.0 |
| FGFR3 [K650M] | 97.3 |
| FGFR3 | 95.8 |
| FGFR2 | 94.1 |
| FGFR4 | 91.8 |
| TNK1 | 89.5 |
| FGFR1 | 69.8 |
| FGFR3 [V555M] | 69.3 |
| FGFR2 [V565I] AKA | 68.6 |
| FGFR2 [V564I] | |
| FGFR3 [V555L] | 68.5 |
| LOK (AKA STK10) | 64.6 |
| FGFR1 [V561M] | 54.1 |
| FGFR4 [V550L] | 44.7 |

AKA = also known as;
FGFR = fibroblast growth factor receptor;
LOK = lymphocyte-oriented kinase;
n = number;
STK10 = serine threonine kinase 10;
TNK1 = tyrosine kinase, non-receptor 1

### Conclusion

In biochemical assays, Compound 1 was a potent inhibitor of FGFR wild type and mutant kinases. In addition, kinome screening in a broad panel of human kinases showed that it had minimal activity towards kinases outside of the FGFR kinase family. These studies demonstrate that Compound 1 is a potent inhibitor of the FGFR family kinases and is selective across the human kinome.

### Example 2: Compound 1 Inhibits Cell Proliferation in Cell Culture

The ability of Compound 1 to inhibit cellular FGFR kinase was tested across a range of assay formats: NanoBRET target engagement, pharmacodynamic biomarker modulation, and tumor cell growth inhibition.

### Cellular FGFR Target Engagement

Intracellular target engagement was demonstrated in a proximity-based assay by measuring energy transfer from a bioluminescent protein donor (NanoLuc fusion) to a fluorescent probe (NanoBRET tracer) in HEK-293 cells. The NanoBRET assay measured the apparent affinity of Compound 1 by competitive displacement of the tracer, which is reversibly bound to the fusion protein in live cells. As shown in Table 4, Compound 1 had target engagement half-maximal inhibitory concentration (IC₅₀) values of 13.7 nM, 7.3 nM, 25.8 nM, and 24.1 nM in FGFR1, FGFR2, FGFR3, and FGFR4 wild type proteins, respectively. NanoBRET IC₅₀ values for FGFR2 mutations including K659M, L617V, N549H (AKA FGFR2-N550H), N549K, and V565F (AKA FGFR2-V564F) were 51.3 nM, 21.7 nM, 6.8 nM, 27.5 nM, and 13.4 nM, respectively. FGFR3 mutations in G697C and V555M had NanoBRET IC₅₀ values of 32.3 nM and 71.6 nM, respectively.

TABLE 4

| Cellular Target | KIN-3248 |
|---|---|
| Engagement | IC₅₀ (nM) |
| FGFR1 | 13.7 |
| FGFR2 | 7.3 |
| FGFR2 [K660M] AKA | 51.3 |
| FGFR2 [K659M] | |
| FGFR2 [L618V] AKA | 21.7 |
| FGFR2 [L617V] | |
| FGFR2 [N550H] AKA | 6.8 |
| FGFR2 [N549H] | |
| FGFR2 [N550K] AKA | 27.5 |
| FGFR2 [N549K] | |
| FGFR2 [V565F] AKA | 13.4 |
| FGFR2 [V564F] | |
| FGFR3 | 25.8 |
| FGFR3 [G697C] | 32.3 |
| FGFR3 [V555M] | 71.6 |
| FGFR4 | 24.1 |

AKA = also known as

Inhibition of pERK in FGFR Dysregulated Human Cancer Cells

Compound 1 demonstrated a range of cellular activity across human FGFR-altered cancer models as determined by MAPK pathway inhibition read out by pERK biomarker pharmacodynamic modulation after 1 hour of treatment in multiple independent experiments, as shown in Table 5. In FGFR2 amplified SNU-16 and KATO-II human gastric cancer cells, pERK EC₅₀ values were 1.26 nM and 2.58 nM, respectively. RT-112 and RT-4 FGFR3-TACC3 fusion-bearing bladder carcinoma cells had pERK EC₅₀ values of 3.02 nM and 2.77 nM, respectively. SW-780 FGFR3-BAIAP2L fusion-bearing bladder carcinoma cells had mean pERK EC₅₀ values of 2.68.

TABLE 5

| | | PERK | | |
|---|---|---|---|---|
| Cell Line | FGFR Alteration | EC₅₀ (nM) | SEM | n |
| SNU-16 | FGFR2 amplification | 1.26 | 0.12 | 4 |
| KATO-III | FGFR2 amplification | 2.58 | 0.48 | 3 |
| RT-112 | FGFR3-TACC3 | 3.02 | 0.33 | 4 |
| RT-4 | FGFR3-TACC3 | 2.77 | 0.33 | 4 |
| SW-780 | FGFR3-BAIAP2L1 | 2.68 | 0.45 | 4 |

BAIAP2L1 = brain-specific angiogenesis inhibitor 1-associated protein 2-like 1;
FGFR = fibroblast growth factor receptor;
FGFR2 = fibroblast growth factor receptor 2;
FGFR3 = fibroblast growth factor receptor 3;
EC₅₀ = half-maximal effective concentration;
n = number;
pERK = phosphorylated extracellular signal-related kinase;
SEM = standard error of the mean;
TACC = transforming acidic coiled-coil;
Thr202/Tyr204 = threonine 202/tyrosine 204

### Inhibition of FGFR2 Autophosphorylation in FGFR2 Amplified Human Cancer Cells

Compound 1 inhibition of the Tyr653/654 autophosphorylation sites of FGFR2 was assessed in 2 FGFR2-amplified human cancer cell lines in multiple independent experiments. Cellular activity was determined by modulation of phosphorylation of FGFR2 after 2 hours of inhibitor treatment and measured in a Meso Scale Discovery assay specific for phosphorylated Tyr653/654, as shown Table 6. Compound 1 demonstrated inhibition of cellular pFGFR2 with mean EC₅₀ values of 3.01 nM and 5.53 nM in the SNU-16 and KATO-III gastric cancer cell lines, respectively.

TABLE 6

| | | pFGFR2 | | |
|---|---|---|---|---|
| Cell Line | FGFR Alteration | EC₅₀ (nM) | SEM | n |
| SNU-16 | FGFR2 amplification | 3.01 | 0.20 | 3 |
| KATO-III | FGFR2 amplification | 5.53 | 0.33 | 3 |

EC₅₀ = half-maximal effective concentration;
FGFR = fibroblast growth factor receptor;
n = number;
pFGFR2 = phosphorylated fibroblast growth factor receptor 2;
SEM = standard error of the mean;
Tyr653/654 = tyrosine 653/654

### Inhibition of FGFR Dysregulated Human Cancer Cell Proliferation

Compound 1 inhibition of cellular proliferation was evaluated across a panel of human FGFR-dysregulated cancer models and measured by CellTiter-Glo (CTG) following 5 days of Compound 1 treatment in multiple independent experiments. As shown in Table 7, Compound 1 EC₅₀ values were 3.89 nM and 5.19 nM in FGFR2 amplified gastric SNU-16 and KATO-III cells. FGFR3 fusion-expressing bladder carcinoma cell lines RT-112, RT-4, and SW-780 had cellular EC₅₀ values of 4.14 nM, 5.96 nM, and 4.14 nM, respectively.

TABLE 7

| | | 5-Day CTG | | |
|---|---|---|---|---|
| Cell Line | FGFR Alteration | EC50 (nM) | SEM | n |
| SNU-16 | FGFR2 amplification | 3.89 | 0.38 | 11 |
| KATO-III | FGFR2 amplification | 5.19 | 0.48 | 11 |
| RT-112 | FGFR3-TACC3 | 4.14 | 0.48 | 15 |
| RT-4 | FGFR3-TACC3 | 5.96 | 0.74 | 12 |
| SW-780 | FGFR3-BAIAP2L1 | 4.14 | 0.31 | 10 |

BAIAP2L1 = brain-specific angiogenesis inhibitor 1-associated protein 2-like 1;
CTG = CellTiter-Glo;
EC₅₀ = half-maximal effective concentration;
FGFR = fibroblast growth factor receptor;
n = number;
SEM = standard error of the mean;
TACC = transforming acidic coiled-coil

### Conclusions

Compound 1 demonstrated cellular target engagement of wild type and mutant FGFR kinase family proteins, as well as cellular activity across a panel of FGFR2- and FGFR3-dysregulated human cancer cell models. Compound 1 cellular target engagement was established in wild type FGFR1, FGFR2, FGFR3, and FGFR4, as well as in FGFR2 and FGFR3 proteins containing known secondary kinase domain resistance mutations in the gatekeeper and molecular brake residues. Compound 1 inhibition of endogenous FGFR in the five human tumor cell lines tested had EC₅₀ values ranging from 1 to 6 nM. Taken together, these findings indicate that Compound 1 potently inhibits growth and FGFR signaling in human FGFR-driven cancer cells and binds clinically relevant mutant FGFR proteins.

### Example 3: Determination of Anti-Proliferative Activity in a Xenograft Model

Compound 1, a novel, next-generation, irreversible, small molecule pan-FGFR inhibitor, inhibits clinically observed secondary mutations known to drive resistance to the approved FGFR kinase inhibitors. Preclinical studies indicate that Compound 1 is potent against FGFR2 and FGFR3 gatekeeper (FGFR2-V565F (also known as FGFR2-V564F) and FGFR3-V555M, respectively), molecular brake (FGFR2-N550X (also known as FGFR2-N549X)), and activation loop (FGFR3-K650M) mutations, amongst others, with low nanomolar biochemical and cellular target engagement half maximal inhibitory concentration (IC₅₀) values. As such, compound 1 has the potential to not only address on-target resistance mutations in patients with FGFR-driven tumors that have progressed on first-generation FGFR inhibitors, but may also extend duration of response in the front-line setting. Here, the in vivo tolerability and antitumor activity of Compound 1 were evaluated in FGFR2- and FGFR3-driven human cancer cell line-derived xenograft models.

### Study Design

Groups and treatments were started when the mean tumor volume reached approximately 200-250 mm³. Mice were assigned to respective groups based on their starting tumor volume and body weight such that the average values for both parameters were balanced for each treatment group. The groups and treatments for evaluation of antitumor activity of Compound 1 in FGFR-driven human cancers (RT-112 and SNU-16) are shown in Table 8.

TABLE 8

| | | Number | Dose | | | | |
|---|---|---|---|---|---|---|---|
| Group | | of | (mg/ | Volume | | Regi- | Dura- |
| Number | Drug | Animals | kg) | (μL/g) | Route | men | tion |
| 1 | Vehicle | 9 | 0 | 10 | PO | BID | 21 days |
| 2 | Compound 1 | 9 | 2 | 10 | PO | QD | 21 days |
| 3 | Compound 1 | 9 | 5 | 10 | PO | QD | 21 days |
| 4 | Compound 1 | 9 | 15 | 10 | PO | QD | 21 days |

FGFR = fibroblast growth factor receptor;
PO = oral;
QD = once daily

### Results

### I. Human RT-112 Urinary Bladder Transitional Cell Carcinoma Xenograft Model

The antitumor activity of Compound 1 was evaluated in the human RT-112 urinary bladder transitional cell carcinoma xenograft model harboring a FGFR3-TACC3 fusion. Treatment with Compound 1 (2, 5, or 15 mg/kg) was initiated when tumor volumes reached approximately 200-250 mm³ (actual mean tumor volume for all groups was 264 mm³) and was continued once daily (QD) for 3 weeks.

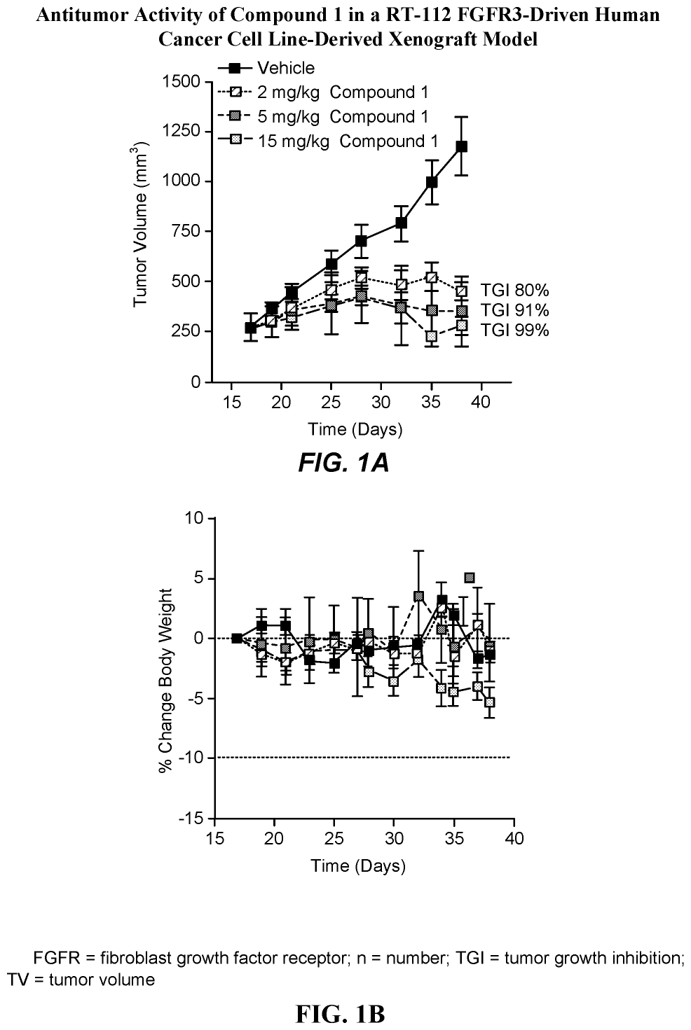

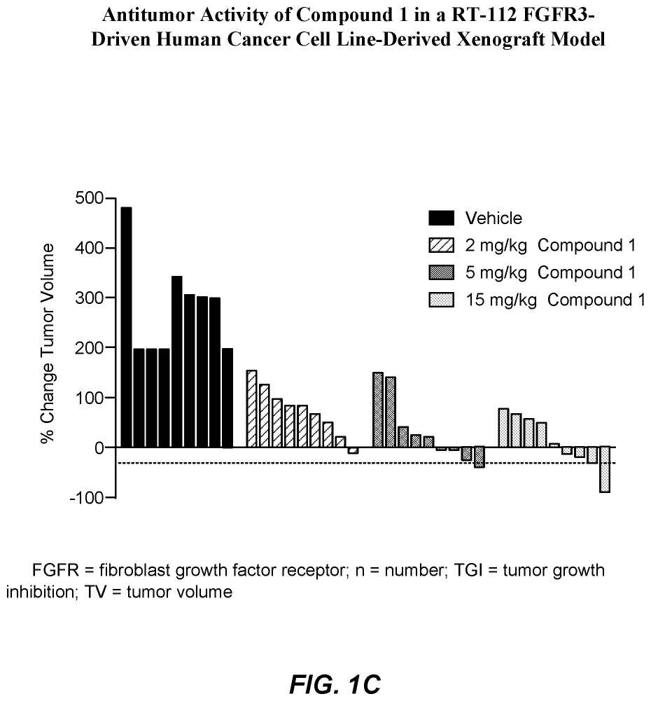

Compound 1 treatment administered PO and QD in mice bearing RT-112 xenografts resulted in AUCₗₐₛₜ values of 1,320-9,760 h*ng/mL over a 24-hour period following the last dose. Dose-dependent inhibition of tumor growth relative to control (vehicle-treated) tumors was observed with Compound 1 treatment (FIG. 1A). All of the tested doses were well tolerated, with some weight loss observed in animals treated with 15 mg/kg (average weight loss of 5.4%; FIG. 1B). Three animals in the 15 mg/kg cohort lost >10% of their body weight during the course of treatment but recovered by end of study.

Waterfall plots of individual tumor response, defined as change in tumor volume from baseline, and mean TGI by treatment group are presented in FIG. 1C and Table 9, respectively. Statistically significant reductions in RT-112 tumor growth were achieved at all tested doses. The average TGI achieved with 2 mg/kg, 5 mg/kg, and 15 mg/kg daily doses was 80%, 91%, and 99%, respectively (p<0.0001; FIG. 1C and Table 9), with 4 of 9 animals (44%) in each of the latter 2 groups exhibiting tumor regression. Table 9 provides a summary of the results of FGFR3-driven bladder cancer (RT-112) xenograft tumor growth inhibition by Compound 1.

TABLE 9

| | Baseline TV | Final TV | ΔT/ΔC | TGI | |
|---|---|---|---|---|---|
| Treatment | (mm³)^{a} | (mm³)^{a} | (%)^{b} | (%)^{c} | P-value^{d} |
| Vehicle | 264 ± 28 | 1171 ± 142 | — | — | — |
| 2 mg/kg QD | 264 ± 26 | 446 ± 49 | 20 | 80 | 0.0001 |
| 5 mg/kg QD | 264 ± 25 | 343 ± 57 | 9 | 91 | <0.0001 |
| 15 mg/kg QD | 264 ± 28 | 274 ± 48 | 1 | 99 | <0.0001 |

— not applicable;
C = control;
FGFR = fibroblast growth factor receptor;
QD = once daily;
RM = repeated measures;
SEM = standard error of the mean;
T = treated;
TGI = tumor growth inhibition;
TV = tumor volume
^{a}Mean ± SEM.
^{b}ΔT/ΔC = (TV_{f} − TV₀)_{treated}/(TV_{f} − TV₀)_{vehicle} × 100% where TV_{f} = final TV (at end of treatment) and TV₀ = initial TV (at beginning of treatment).
^{c}TGI = (1 − T/C) × 100%.
^{d}P-value as determined by two-way RM analysis of variance followed by Turkey’s post hoc comparison of the means.

### II. Human SNU-16 Gastric Cancer Xenograft Model

The antitumor activity of Compound 1 was next evaluated in a human xenograft model exhibiting FGFR2 gene amplification as well as low levels of FGFR2 fusions, including FGFR2-PDHX. SNU-16 gastric cancer cell-line derived xenografts were similarly treated with 2 mg/kg, 5 mg/kg, and 15 mg/kg daily of compound 1 for 3 weeks. Treatment was initiated when tumor volumes were approximately 200-250 mm³ (actual mean TV for all groups was 262 mm³).

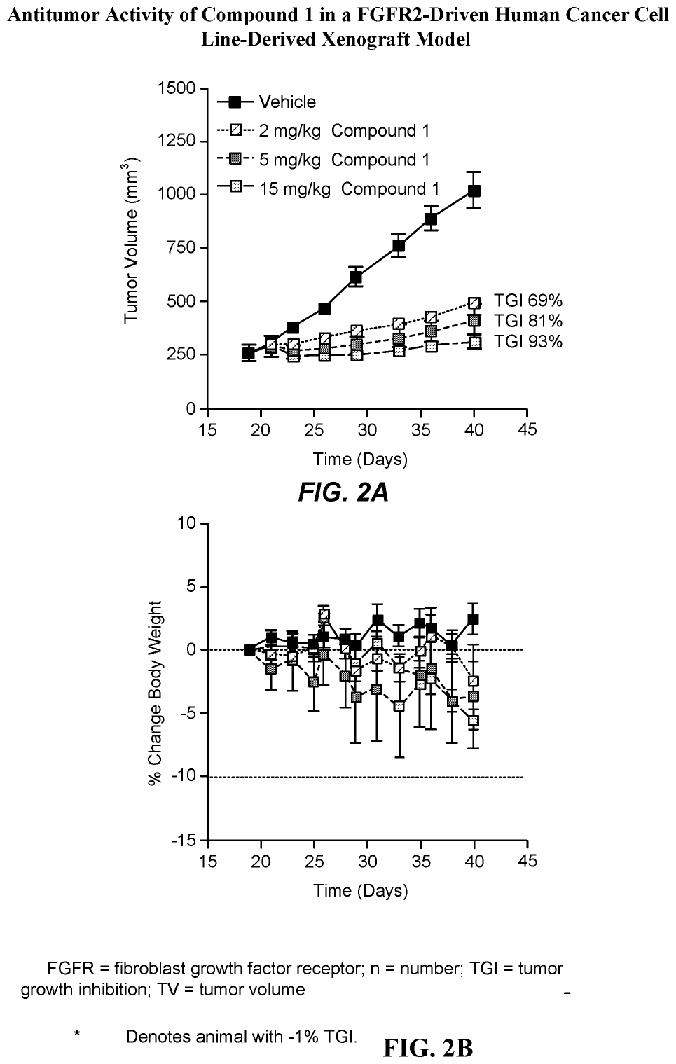

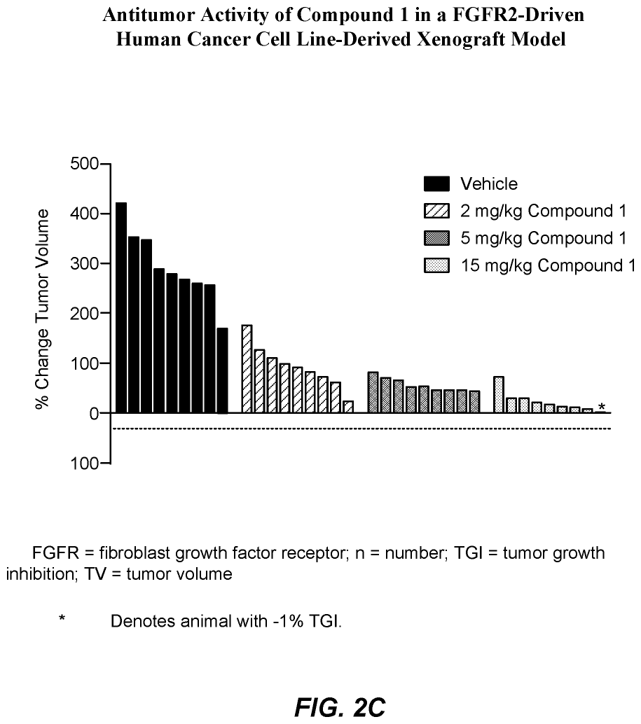

Compound 1 treatment administered PO and QD in mice bearing SNU-16 xenografts resulted in AUCₗₐₛₜ values of 1,090-9,760 h*ng/mL over a 24-hour period following the last dose. Dose-dependent inhibition of SNU-16 tumor growth relative to control (vehicle-treated) tumors was observed with Compound 1 treatment (FIG. 2A). All tested doses and schedules were well tolerated, as indicated by a lack of significant body weight changes in on-treatment animals (FIG. 2B).

Waterfall plots of individual tumor response and mean TGI by treatment group are presented in FIG. 2C and Table 10, respectively. Statistically significant reductions in SNU-16 tumor growth were achieved at all tested doses. The average TGI achieved with 2 mg/kg, 5 mg/kg, and 15 mg/kg daily doses was 69%, 81%, and 93%, respectively (p<0.0001; FIG. 2, Table 10). Tumor regression was observed in one animal in the 15 mg/kg QD cohort. Table 10 provides a summary of the results of FGFR2-driven gastric cancer (SNU-16) xenograft tumor growth inhibition by Compound 1.

TABLE 10

| | Baseline TV | Final TV | ΔT/ΔC | TGI | |
|---|---|---|---|---|---|
| Treatment | (mm³)^{a} | (mm³)^{a} | (%)^{b} | (%)^{c} | P-value^{d} |
| Vehicle | 262 ± 16 | 1023 ± 83 | — | — | — |
| 2 mg/kg QD | 262 ± 14 | 497 ± 22 | 31 | 69 | <0.0001 |
| 5 mg/kg QD | 262 ± 12 | 410 ± 20 | 19 | 81 | <0.0001 |
| 15 mg/kg QD | 262 ± 16 | 313 ± 13 | 7 | 93 | <0.0001 |

— not applicable;
C = control;
FGFR = fibroblast growth factor receptor;
QD = once daily;
RM = repeated measures;
SEM = standard error of the mean;
T = treated;
TGI = tumor growth inhibition;
TV = tumor volume
^{a}Mean ± SEM.
^{b}ΔT/ΔC = (TV_{f} − TV₀)_{treated}/(TV_{f} − TV₀)_{vehicle} × 100% where TV_{f} = final TV (at end of treatment) and TV₀ = initial TV (at beginning of treatment).
^{c}TGI = (1 − T/C) × 100%.
^{d}P-value as determined by 2-way RM analysis of variance followed by Turkey’s post hoc comparison of the means.

### Conclusions

Dose-dependent inhibition of FGFR2- and FGFR3-driven human cancer cell line-derived xenograft growth was observed with daily Compound 1 treatment (2-15 mg/kg). RT-112 (FGFR3-TACC3 fusion-positive) and SNU-16 (FGFR2 amplified and FGFR2-PDHX fusion-positive) xenografts exhibited 80-99% and 69-93% mean TGI, respectively (p<0.0001). Body weight loss was noted at the highest Compound 1 dose (15 mg/kg) in the former, but not the latter model, and not with other tested doses. Taken together, PO administration of Compound 1 is generally well-tolerated and efficacious in mouse xenograft models of FGFR-driven human cancers.

### Example 4: Use of Compound 1 in a Human Clinical Trial

Title: A Phase 1/1b, Open-label, Multicenter, Two-part Study to Investigate the Safety, Tolerability, Pharmacokinetics, Pharmacodynamics, and Anti-tumor Activity of Compound 1 in Participants with Advanced Tumors Harboring FGFR1, FGFR2 and/or FGFR3 Gene Alterations
Investigational Drug: Compound 1
Study Phase: Phase 1/1b
Indication: Advanced Tumors Harboring FGFR1, FGFR2 and/or FGFR3 Gene Alterations

### Introduction

This is a first in human (FIH), two-part, open-label, multicenter, dose escalation and dose expansion study designed to evaluate the safety, tolerability, pharmacokinetics (PK), pharmacodynamics (PD), and anti-tumor activity of Compound 1, a next-generation, irreversible, small molecule pan-fibroblast growth factor receptor (FGFR) inhibitor. Further, this study will determine a recommended Phase 2 dose (RP2D) of Compound 1 for further clinical development and assess the objective response to Compound 1 therapy in participants with advanced tumors harboring pertinent FGFR1, FGFR2 and/or FGFR3 gene alterations.

### Study Objectives

The primary objectives of the Part A, Dose Escalation portion of the study are to determine the safety and tolerability of oral administration of Compound 1 including dose-limiting toxicities (DLT) in participants with advanced tumors harboring FGFR1, FGFR2 and/or FGFR3 gene alterations and to identify the maximum tolerated dose (MTD) and/or the RP2D of Compound 1 for further clinical development.

The primary objective of the Part B, Dose Expansion portion of the study is to assess preliminary evidence of the anti-tumor activity of Compound 1 in participants with advanced tumors harboring FGFR1, FGFR2 and/or FGFR3 gene alterations (including intrahepatic cholangiocarcinoma [ICC], urothelial carcinoma [UC], and other solid tumors, as appropriate).

The secondary objective is to characterize the PK of Compound 1.

The exploratory objectives include additional characterization of exposure response relationships for efficacy and safety and potential Compound 1 metabolites; to assess on-target PD modulation by Compound 1 and evaluate potential biomarkers for response/resistance to Compound 1 in blood samples and/or tumor biopsies.

### Study Design

The study will be conducted in 2 parts: Part A, Dose Escalation, and Part B, Dose Expansion. Part A is aimed at evaluating the safety, tolerability, PK, and PD of Compound 1 and determining the MTD of once-daily (QD) dosing schedule in participants with advanced tumors harboring FGFR1, FGFR2 and/or FGFR3 gene alterations using a modified Bayesian optimal interval (BOIN) design. The Dose Expansion part of the study (Part B) can open once the MTD and/or a biologically active dose (e.g., RP2D of Compound 1 for further clinical development) has been determined in Part A. Part A will consist of up to approximately 45 participants and Part B will consist of approximately 75 participants, including at least 3 cohorts with advanced tumors harboring FGFR1, FGFR2 and/or FGFR3 gene alterations (i.e., ICC, UC, and all other advanced tumors). Different dosing schedules may be used in Part B based on clinical and PK data from Part A but will be limited to those having a lesser dose intensity than the 28-day daily schedule. The DLT evaluation period will be 28 days.

Administration of Compound 1 to participants in Part A and Part B may continue until evidence of disease progression, intolerance to study medication, unacceptable toxicity, initiation of new systemic therapy for cancer, withdrawal of consent, Investigator/Sponsor decision, or death.

### Part A (Dose Escalation):

Part A will follow a modified BOIN dose escalation schema to identify the MTD and/or RP2D of Compound 1 in participants with advanced tumors harboring FGFR1, FGFR2 and/or FGFR3 gene alterations. The target DLT rate for the MTD is defined as 30% of participants at a dose level experiencing a DLT during the 28-day DLT evaluation period.

In Part A, participants with advanced tumors harboring FGFR2 and/or FGFR3 gene alterations will be included, subject to meeting all protocol-defined eligibility requirements.

Compound 1 will be administered as an oral dose QD in 28-day treatment cycles to participants with advanced tumors harboring FGFR1, FGFR2 and/or FGFR3 gene alterations. Alternate dose schedules may be included depending on the study data and recommended by the Dose Review Committee (DRC).

The study will start with a cohort size of 3. The starting dose, Dose Level 1 (DL1), will be 5 mg (see Table 11) by comparing the observed DLT rate at the current dose level with a fixed, prespecified dose escalation boundary of 0.197 (λₑ) and a de-escalation boundary of 0.298 (λ_{d}). The tentative Compound 1 dose levels for Part A dose escalation. The decision to escalate, de-escalate, eliminate, or maintain the current dose can also be made based on the observed number of DLTs relative to the number of participants treated at the current dose level Table 2). “Eliminate” means eliminating the current and higher doses from the study to prevent treating any future patients at these dose levels because they are overly toxic. When the lowest dose is eliminated, the trial is stopped without selecting an MTD.

TABLE 11

| Dose Level | Target Daily Dose |
|---|---|
| 1 | 5 mg |
| 2 | 10 mg |
| 3 | 20 mg |
| 4 | 30 mg |
| 5 | 40 mg |
| 6 | 50 mg |

*Dose escalation step size will be capped at 100%. Number of participants enrolled in a cohort will depend on if a DLT occurs during that dosing cohort. The maximum cohort size will be 9 participants.

TABLE 12

| | Number of participants treated | | | | | | | | |
|---|---|---|---|---|---|---|---|---|---|
| | 1 | 2 | 3 | 4 | 5 | 6 | 7 | 8 | 9 |
| Escalate if # | 0 | 0 | 0 | 0 | 0 | 1 | 1 | 1 | 1 |
| of DLT≤ | | | | | | | | | |
| De-escalate if # | 1 | 1 | 2 | 2 | 2 | 2 | 3 | 3 | 3 |
| of DLT≥ | | | | | | | | | |
| Stay if # DLT= | NA | NA | 1 | 1 | 1 | NA | 2 | 2 | 2 |
| Eliminate if # | NA | NA | 3 | 3 | 3 | 4 | 4 | 4 | 5 |
| of DLT≥ | | | | | | | | | |

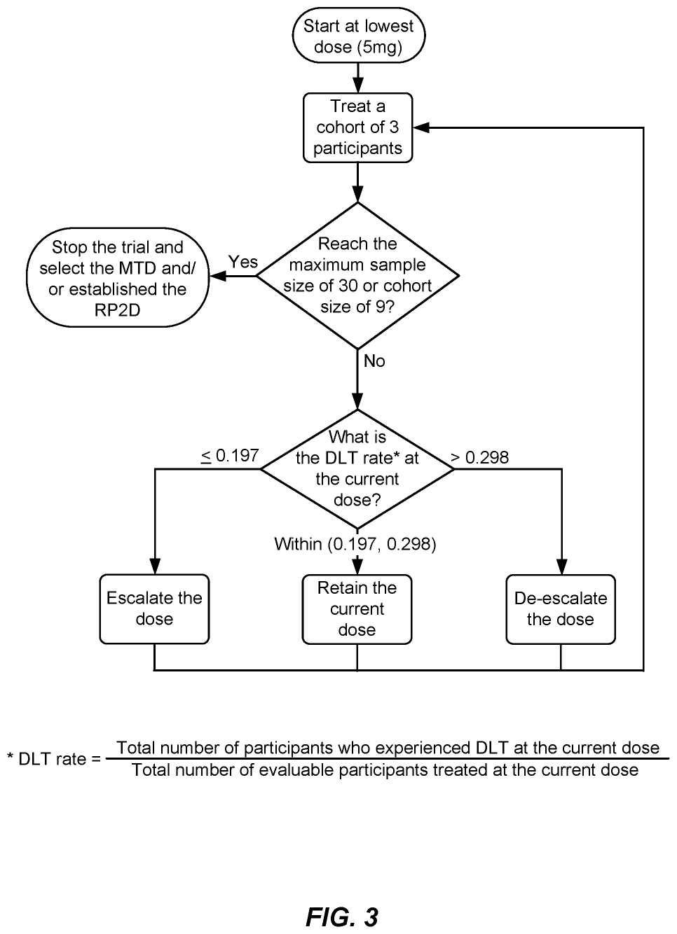

Dose escalation will continue to the next higher dose following the modified BOIN decision rules outlined in Table 12 and FIG. 3 until the planned sample size (n=30) is reached or the maximum cohort size is reached (n=9). Tentative Compound 1 dose levels are indicated in Table 11. Intermediate doses may be recommended as appropriate.

A Dose Review Committee (DRC) consisting of Investigator and Sponsor representatives will review available safety, PK, and PD data prior to initiating enrollment at the next dose level. Specific step sizes may be determined with reference to the modified BOIN design recommendations, potentially with additional supplementary Bayesian modeling. The dose escalation and de-escalation rules outlined above will also apply to any intermediate dose levels that are studied. The DRC will determine the MTD and/or RP2D of Compound 1 for further clinical development when sufficient safety, efficacy, and PK/PD data are available.

Intra-participant dose escalation and backfill are allowed in Part A of this study at the discretion of the Sponsor.

### Part B (Dose Expansion):

Part B will evaluate the anti-tumor activity of Compound 1 at the recommended dose of Compound 1 for cohort expansion determined from Part A in the following cohorts (approximately 25 participants/cohort):
Cohort 1: Participants with advanced ICC with FGFR2 gene alterations
Cohort 2: Participants with advanced UC with FGFR2 and/or FGFR3 gene alterations
Cohort 3: Participants with advanced tumor (other than ICC or UC) with FGFR1, FGFR2 and/or FGFR3 gene alterations

Enrollment of participants in the three Dose Expansion cohorts may occur concurrently, but not in Part A. There will be a table of acceptable gene alterations provided for guidance.

For Cohorts 1 and 2, a Simon's 2-stage optimal design is planned.

In Cohort 3, enrollment of participants with various tumor types that harbor FGFR2 and/or FGFR3 gene alterations will be monitored during the course of this study; enrollment of specific tumor types may be restricted to ensure broad representation of various advanced tumors in this cohort.

### Study Endpoints

### Primary

Safety endpoints include the following:
Incidence of dose limiting toxicity (DLTs)
Incidence of Adverse Events (AEs), including treatment-emergent adverse events (TEAEs) and treatment-related adverse events (TRAEs)
Clinically significant changes in vital signs, physical examinations, 12-lead electrocardiograms (ECGs), and clinical laboratory tests

Efficacy will be measured by the following:
Objective response rate (ORR) defined as the rate of partial responses (PR) plus complete responses (CR) according to Response Evaluation Criteria in Solid Tumors (RECIST) v1.1
Disease control rate (DCR)
Duration of response (DOR)
Progression-free survival (PFS)

### Secondary

PK parameters of Compound 1 including, but not limited to, maximum observed plasma concentration (Cₘₐₓ), time to achieve Cₘₐₓ (tₘₐₓ), and area under the plasma concentration-time curve (AUC).

### Exploratory

Compound 1 exposure-safety and exposure-efficacy relationships
Overall survival (OS)
Duration of stable disease
Characterization of potential metabolites of Compound 1 in plasma and urine
In the expansion cohort (Part B), quality of life PROs may be incorporated (e.g., 5Q-ED-5L).
To evaluate pharmacodynamic relationships and characterize potential resistance mechanisms by molecular and/or genetic analysis of biomarkers including, but not limited to, phosphorous levels, FGF23 levels, genomic analysis, gene expression profiling (GEP), and FGFR pathway modulation in blood and/or tumor biopsy samples.

### Sample Size

### Part A (Dose Escalation):

Up to approximately 45 participants will be enrolled in Part A of the study.

Approximately 30 of the 45 participants will be enrolled following the modified BOIN design. Up to 15 additional participants may be enrolled as backfills to further characterize the MTD and/or RP2D.

### Part B (Dose Expansion):

Approximately 75 participants will be enrolled in Part B of the study, which will include the following cohorts (approximately 25 participants/cohort):
Cohort 1: Participants with advanced ICC with FGFR2 gene alterations
Cohort 2: Participants with advanced UC with FGFR2 and/or FGFR3 gene alterations
Cohort 3: Participants with advanced tumor (other than ICC or UC) with FGFR1, FGFR2 and/or FGFR3 gene alterations

### Summary of Participant Eligibility Criteria

Adult participants (≥18 years of age or ≥age of majority in local jurisdictions) with histologically or cytologically confirmed diagnosis of advanced-stage malignancy will be eligible for this study. Part A, Dose Escalation, will enroll participants with any type of advanced tumors harboring FGFR1, FGFR2 and/or FGFR3 gene alterations. Part B, Dose Expansion, will enroll participants with advanced ICC with FGFR2 gene alterations, advanced UC with FGFR2 and/or FGFR3 gene alterations, or advanced tumor (other than ICC or UC) with FGFR1, FGFR2 and/or FGFR3 gene alterations. Participants must have either received prior standard of care therapy (including agents approved in local jurisdictions) appropriate for their tumor type and stage of disease or, in the opinion of the Investigator, be unlikely to tolerate or to derive clinically meaningful benefit from standard of care therapy. Participants with a history and/or current evidence of non-tumor related alteration of calcium-phosphorous homeostasis, a history and/or current evidence of clinically significant ectopic mineralization/calcification, or a history and/or current evidence of a clinically significant retinal disorder will be excluded from participating in this study.

Enrollment will be restricted to participants with advanced tumors harboring FGFR1, FGFR2 and/or FGFR3 gene alterations that have been confirmed by previous genomic analysis of tumor tissue or ctDNA conducted in a Clinical Laboratory Improvement Amendments (CLIA)-certified laboratory (in United States [US]) or in accordance with local regulatory requirements (in other countries). Once consented, the participants will provide a medical history and undergo screening safety tests to confirm all eligibility requirements of the study have been met. Participants will provide an archived tumor tissue specimen (formalin-fixed paraffin embedded [FFPE] specimen) obtained within the last 5 years (if available) and will undergo mandatory pre-treatment tumor biopsy, if medically feasible.

### Study Sites/Locations

The study will be conducted globally at approximately 10 sites for Part A and approximately 30 sites for Part B.

### Study Duration

The estimated duration of this study is approximately 4 years.

### Treatment Duration

Participants will receive Compound 1 in 28-day cycles until evidence of disease progression, intolerance to study medication, unacceptable toxicity, initiation of new systemic therapy for cancer, withdrawal of consent, Investigator decision, Sponsor decision, or death.

### Statistical Considerations

The modified BOIN design is used in Part A of the study with a target DLT rate for the MTD of 25%. Once the Part A Dose Escalation is complete, an isotonic regression analysis will be performed to identify the MTD and/or the RP2D of Compound 1 for further clinical development. Summary of observed DLTs across all dose levels will be provided along with summaries of AEs, and serious adverse events (SAEs).

Safety analysis including analysis of all AEs, laboratory test values, and vital signs will include all participants who have received at least one dose of Compound 1 in both parts of the study.

Efficacy analysis will focus on participants enrolled in Part B. However, participants receiving the same dose as in Part B may be pooled into the appropriate disease cohorts as sensitivity analyses. For the ORR endpoint, the Clopper-Pearson 95% confidence intervals (CIs) will be provided. DOR will be calculated among responders (CR and PR). PFS, OS and duration of SD will be analyzed using the Kaplan-Meier method with graphical displays of the Kaplan-Meier curves.

What is claimed is:

1. A method of treating a cancer in a patient in need thereof, comprising administering to the patient 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof.

2. A method of treating a cancer in a patient in need thereof, comprising administering to the patient a pharmaceutical composition comprising 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, and at least one pharmaceutically acceptable excipient.

3. A method of treating a cancer in a patient in need thereof, comprising administering to the patient:
(a) a composition comprising 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, and
(b) at least one oncology therapeutic selected from the group consisting of an mTOR inhibitor, a MAPK/PI3K inhibitor, an immune checkpoint inhibitor, an EGFR kinase inhibitor or antibody, a HER2 kinase inhibitor, an estrogen receptor antagonist, an androgen receptor antagonist, a CDK kinase inhibitor, an ALK receptor tyrosine kinase inhibitor, a ROS receptor tyrosine kinase inhibitor, a NTRK receptor tyrosine kinase inhibitor, or a chemotherapy regimen.

4. The method of claim 1, 2, or 3, wherein the cancer is characterized by the presence of at least one oncogenic FGFR1, FGFR2 or FGFR3 gene alteration.

5. The method of claim 4, wherein the oncogenic FGFR alteration is selected from the group consisting of:
FGFR2 [K660M];
FGFR2 [K659M];
FGFR2 [L618V];
FGFR2 [L617V];
FGFR2 [N550H];
FGFR2 [N549H];
FGFR2 [N550K];
FGFR2 [N549K];
FGFR2 [V565F];
FGFR2 [V564F];
FGFR2 [N550S/T];
FGFR2 [N549S/T];
FGFR3 [G697C];
FGFR3 [V555M];
FGFR3 [K650E];
FGFR3 [N540K/S];
FGFR3 [K650M]; and
FGFR1 [V561M]; or a combination thereof.

6. The method of claim 4, wherein the oncogenic FGFR alteration is FGFR2 amplification, FGFR3-TACC3, or FGFR3-BAIAP2L1.

7. The method of claim 1, 2 or 3, wherein the cancer is characterized as having a wild type FGFR2 or FGFR3.

8. The method of any one of the preceding claims wherein the patient is selected due to a FGFR2 or FGFR3 fusion or rearrangement as detected by an FDA-approved test.

9. The method of any one of the preceding claims, wherein the cancer is a solid tumor.

10. The method of any one of the preceding claims, wherein the cancer is selected from the group consisting of bladder cancer, urinary bladder carcinoma, urothelial carcinoma, urothelial cancer, renal cell carcinoma, prostate cancer, double negative prostate, castration-resistant prostate cancer, gastric carcinoma, gastric cancer, gastroesophageal junction adenocarcinoma, hepatocellular carcinoma, cholangiocarcinoma, intrahepatic cholangiocarcinoma, pancreatic adenocarcinoma, pancreatic cancer breast cancer, HER2(−)/ER(+) breast cancer, HER2(−)/ER(+)/PR(+) breast cancer, non-Hodgkin lymphoma, acute myeloid leukemia, myeloproliferative neoplasm, polycythemia vera, essential thrombocythemia, primary myelofibrosis, multiple myeloma, glioblastoma, glioma, astrocytoma, anaplastic astrocytoma, medulloblastoma, oligodendroglioma, anaplastic oligodendroglioma, meningioma, lung cancer, and non-small cell lung cancer.

11. The method of claim 10, wherein the cancer is selected from bladder cancer, urinary bladder carcinoma, urothelial carcinoma, urothelial cancer, cholangiocarcinoma, or intrahepatic cholangiocarcinoma.

12. The method of any one of the preceding claims, wherein the cancer is metastatic.

13. The method of any one of the preceding claims, wherein the method is adjuvant therapy following surgical resection.

14. The method of any one of claims 1-12, wherein the method is neo-adjuvant therapy before surgical resection.

15. The method of any one of claims 1-14, wherein the patient has relapsed after prior therapy.

16. The method of any one of claims 1-14, wherein the patient has acquired resistance to prior therapy.

17. The method of any one of claims 1-14, wherein the patient is refractory to therapy.

18. The method of any one of claims 3-17, wherein the at least one oncology therapeutic is an immune checkpoint inhibitor.

19. The method of claim 18, wherein the immune checkpoint inhibitor is a CTLA-4 inhibitor, a PD-1 inhibitor, or a PD-L1 inhibitor.

20. The method of claim 19, wherein the CTLA-4 inhibitor is ipilimumab.

21. The method of claim 19, wherein the PD-1 inhibitor is spartalizumab, nivolumab, pembrolizumab, or cemiplimab.

22. The method of claim 19, wherein the PD-L1 inhibitor is atezolizumab, avelumab, or durvalumab.

23. The method of any one of claims 3-17, wherein the at least one oncology therapeutic is a CDK inhibitor.

24. The method of claim 23, wherein the CDK inhibitor is a CDK4/6 inhibitor.

25. The method of claim 24, wherein the CDK4/6 inhibitor is palbociclib, abemaciclib, or ribociclib.

26. The method of any one of claims 3-17, wherein the at least one oncology therapeutic is an EGFR kinase inhibitor or antibody.

27. The method of claim 26, wherein the EGFR kinase inhibitor is nazartinib, gefitinib, erlotinib, afatinib, brigatinib, icotinib, neratinib, osimertinib, dacomitinib, or lapatinib.

28. The method of claim 26, wherein the EGFR antibody is cetuximab, panitumumab, zalutumumab, nimotuzumab, or matuzumab.

29. The method of any one of claims 3-17, wherein the at least one oncology therapeutic is an mTOR inhibitor.

30. The method of claim 29, wherein the mTOR inhibitor is rapamycin.

31. The method of any one of claims 3-17, wherein the at least one oncology therapeutic is a MAPK/PI3K inhibitor.

32. The method of claim 31, wherein the MAPK/PI3K inhibitor is binimetinib or copanlisib.

33. The method of any one of claims 3-17, wherein the at least one oncology therapeutic is a HER2 kinase inhibitor.

34. The method of claim 33, wherein the HER2 inhibitor is lapatinib.

35. The method of any one of claims 3-17, wherein the at least one oncology therapeutic is an estrogen receptor antagonist.

36. The method of claim 35, wherein the estrogen receptor antagonist is fulvestrant.

37. The method of any one of claims 3-17, wherein the at least one oncology therapeutic is an androgen receptor antagonist.

38. The method of claim 37, wherein the androgen receptor antagonist is enzalutamide.

39. The method of any one of claims 3-17, wherein the at least one oncology therapeutic is selected from an ALK receptor tyrosine kinase inhibitor, a ROS receptor tyrosine kinase inhibitor, or a NTRK receptor tyrosine kinase inhibitor.

40. The method of any one of claims 3-17, wherein the at least one oncology therapeutic is a chemotherapy regimen.

41. The method of claim 40, wherein the chemotherapy regimen comprises a platinum-based chemotherapy.

42. The method of claim 41, wherein the platinum-based chemotherapy is oxaliplatin, cisplatin, or carboplatin.

43. The method of claim 40, wherein the chemotherapy regimen comprises a gemcitabine regimen.

44. The method of claim 40, wherein the chemotherapy regimen comprises a FOLFOX regimen.

45. The method of claim 40, wherein the FOLFOX regimen comprises folinic acid.

46. The method of claim 40, wherein the FOLFOX regimen comprises 5-fluorouracil.

47. The method of claim 40, wherein the FOLFOX regimen comprises folinic acid and folinic acid.

48. The method of any one of the preceding claims, wherein the 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, or pharmaceutically acceptable salt or solvate thereof, is administered orally.

49. The method of claim 48, wherein the oral administration occurs every other day, once per day, twice per day, or three times per day.
